(19) United States
(12) Patent Application Publication
(10) Pub. No.: US 2024/0024206 A1
(43) Pub. Date: Jan. 25, 2024

(54) SEPARATING MEMBRANE FOR DENTAL SURGERIES AND METHOD FOR MANUFACTURING THE SAME

(71) Applicant: NAN YA PLASTICS CORPORATION, Taipei (TW)

(72) Inventors: TE-CHAO LIAO, TAIPEI (TW); Min-Fan Chung, Taoyuan City (TW); CHIA-JUNG CHANG, TAIPEI (TW); CHING-YAO YUAN, TAIPEI (TW)

(21) Appl. No.: 18/070,405

(22) Filed: Nov. 28, 2022

(30) Foreign Application Priority Data
Jul. 20, 2022 (TW) 111127113

(51) Int. Cl. A61K 6/20; A61K 6/60; A61K 6/69

(52) U.S. Cl. CPC A61K 6/20; A61K 6/60; A61K 6/69

(57) ABSTRACT

A separating membrane for dental surgeries and a method for manufacturing the same are provided. The separating membrane has biodegradability, and includes a biodegradable porous base layer and a hydrophilic substance. The hydrophilic substance is bonded to the biodegradable porous base layer, and is selected from the group consisting of hyaluronic acid, derivatives of the hyaluronic acid, and water-soluble vitamins. An outer surface of the separating membrane has a water contact angle of less than 80°.

### CROSS-REFERENCE TO RELATED PATENT APPLICATION

This application claims the benefit of priority to Taiwan Patent Application No. 111127113, filed on Jul. 20, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a medical membrane, and more particularly to a separating membrane for dental surgeries and a method for manufacturing the same.

### BACKGROUND OF THE DISCLOSURE

A guided bone regeneration (GBR) procedure, which is also referred to as a bone repair operation, is usually performed before a dental implant. This can solve problems caused by having a tooth missing for an extended period of time (e.g., shrinkage of an alveolar bone).

Referring to FIG. 5, during the GBR procedure, a gum tissue G is cut apart, and then bone grafts B are filled in a depression of the alveolar bone, so as to facilitate hyperplasia of bone cells in a tooth ridge R. In order to prevent a growing space for the bone cells from being occupied by the gum tissue G or soft tissues during cell proliferation, a separating membrane F is disposed on the bone grafts B to separate the alveolar bone from the soft tissues. Finally, the gum tissue G is stitched up. Accordingly, the bone cells can grow within a specific space to rebuild the tooth ridge R.

A conventional separating membrane currently available on the market is made from collagen (hereinafter referred to as a collagen membrane). Since physical properties of the collagen membrane are weak, the collagen membrane is likely to rupture after the implant, which causes the artificial bones to fall. Moreover, the collagen membrane is not moldable. In order to completely cover a wound, dentists need to fix the shape of the collagen membrane by sewing or other auxiliary means. Accordingly, the collagen membrane is inconvenient for use due to its weak physical properties.

Therefore, how to improve the physical properties of the conventional separating membrane and increase its convenience of use, so as to overcome the above-mentioned problems, has become one of the important issues to be addressed in the industry.

### SUMMARY OF THE DISCLOSURE

The focus of the present disclosure is to improve water wettability, coverability, and adhesive strength of a separating membrane for dental surgeries. The technical approach adopted in the present disclosure includes: forming a porous structure to serve as a base layer of the separating membrane, and performing a hydrophilization treatment on the base layer by use of a hydrophilic substance. A molecular weight of the hydrophilic substance is between a molecular weight of a small molecule and a molecular weight of a polymer.

In one aspect, the present disclosure provides a separating membrane for dental surgeries, which includes a biodegradable porous base layer and a hydrophilic substance. The hydrophilic substance is bonded to the biodegradable porous base layer, and is selected from the group consisting of hyaluronic acid, derivatives of the hyaluronic acid, and water-soluble vitamins. An outer surface of the separating membrane has a water contact angle of less than 80°.

In one embodiment of the present disclosure, a material of the biodegradable porous base layer includes polylactic acid, and the hydrophilic substance is hyaluronic acid having a molecular weight between 10,000 daltons and 700,000 daltons.

In one embodiment of the present disclosure, a thickness of the biodegradable porous base layer is from 200 μm to 400 μm.

In one embodiment of the present disclosure, the biodegradable porous base layer includes a plurality of polylactic acid fibers adhered with the hydrophilic substance.

In one embodiment of the present disclosure, the separating membrane further includes an outer covering layer. The outer covering layer covers the biodegradable porous base layer, and the hydrophilic substance is present in the outer covering layer.

In one embodiment of the present disclosure, a tensile stress of the separating membrane is from 0.3 MPa to 5 MPa at 25° C. and an absolute humidity of 50%.

In one embodiment of the present disclosure, an adhesive strength of the separating membrane is from 0.3 N to 0.7 N according to the ASTM D3121-2006 standard.

In another aspect, the present disclosure provides a method for manufacturing a separating membrane for dental surgeries, which includes: providing a biodegradable porous base layer; and treating the biodegradable porous base layer with an aqueous solution containing a hydrophilic substance, such that the hydrophilic substance is bonded to the biodegradable porous base layer. The hydrophilic substance is selected from the group consisting of hyaluronic acid, derivatives of the hyaluronic acid, and water-soluble vitamins. An outer surface of the separating membrane has a water contact angle of less than 80°.

In one embodiment of the present disclosure, based on a total weight of the aqueous solution being 100 wt %, a content of the hydrophilic substance is from 25 wt % to 40 wt %.

In one embodiment of the present disclosure, in the step of treating the biodegradable porous base layer with the aqueous solution containing the hydrophilic substance, the biodegradable porous base layer is immersed in the aqueous solution for 30 seconds to 1 minute.

In one embodiment of the present disclosure, after the step of treating the biodegradable porous base layer with the aqueous solution containing the hydrophilic substance is completed, an outer covering layer is formed to cover the biodegradable porous base layer is formed, and the hydrophilic substance is present in the outer covering layer.

In one embodiment of the present disclosure, in the step of providing the biodegradable porous base layer, a plurality of polylactic acid fibers are provided and formed into a layered structure by electrospinning After the step of treating the biodegradable porous base layer with the aqueous solution containing the hydrophilic substance is completed, the plurality of polylactic acid fibers are adhered with the hydrophilic substance.

Therefore, in the separating membrane for dental surgeries and the method for manufacturing the same provided by the present disclosure, by virtue of “the hydrophilic substance being bonded to the biodegradable porous base layer, and being selected from the group consisting of hyaluronic acid, derivatives of the hyaluronic acid, and water-soluble vitamins” and “an outer surface of the separating membrane having a water contact angle of less than 80°,” an immersion and wetting time of the separating membrane can be greatly reduced before use (e.g., before surgical operations), and the fully-wetted separating membrane can have a better moldability, a better coverability, and a better adhesive strength.

More specifically, the separating membrane of the present disclosure can absorb water to achieve a moldable softened state within 5 minutes, so as to be adaptable for different three-dimensional shapes. Furthermore, the separating membrane of the present disclosure can securely adhere to a damaged area (e.g., a bone damaged area) and provide a sufficient growth space for the damaged area, thereby facilitating the repair, regeneration, and integration of the damaged area.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic structural view of a separating membrane for dental surgeries according to the present disclosure;

FIG. 2 is another schematic structural view of the separating membrane for dental surgeries according to the present disclosure;

FIG. 3 is a partial enlarged view of the separating membrane for dental surgeries according to the present disclosure;

FIG. 4 is a flowchart of a method for manufacturing the separating membrane for dental surgeries according to the present disclosure; and

FIG. 5 is a schematic view showing a guided bone regeneration (GBR) procedure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

Unless otherwise stated, the material(s) used in any described embodiment is/are commercially available material(s) or may be prepared by methods known in the art, and the equipment or operation(s) used in any described embodiment is/are conventional equipment or operation(s) generally known in the related art.

In the present disclosure, multiple steps shown in a method flowchart are described in a specific order. However, this does not indicate or imply that these steps must be executed in the specific order or desired results can only be achieved by execution of all the steps. In practice, it is optional to combine two or more steps into one step, or to divide one step into two or more steps.

### First Embodiment

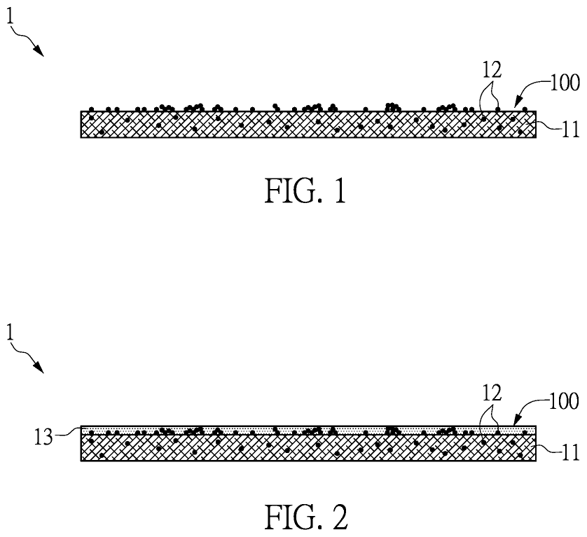

Referring to FIG. 1 and FIG. 2, a first embodiment of the present disclosure provides a separating membrane 1 for dental surgeries, which mainly includes a biodegradable porous base layer 11 and a hydrophilic substance 12. The hydrophilic substance 12 is bonded to the biodegradable porous base layer 11. Accordingly, an outer surface 100 of the separating membrane 1 has a water contact angle of less than 80°, such that an immersion and wetting time of the separating membrane 1 can be greatly reduced before use. Further, the fully-wetted separating membrane 1 can have a better moldability, a better coverability, and a better adhesive strength. In the present disclosure, the water contact angle of the outer surface 100 of the separating membrane 1 is preferably less than 60°, more preferably less than 30°, and most preferably less than 10°.

Specifically, the separating membrane 1 of the present disclosure is formed by performing a hydrophilization treatment on the biodegradable porous base layer 11. In a treatment process, the hydrophilic substance 12 not only adheres to an outer surface of the biodegradable porous base layer 11, but also penetrates into the biodegradable porous base layer 11. The hydrophilization treatment is performed for adhesion of the hydrophilic substance 12. That is, the biodegradable porous base layer 11 is integrated with the hydrophilic substance 12 to form a composite structure (in which microbonds are formed between the hydrophilic substance 12 and the biodegradable porous base layer 11). Accordingly, the biodegradable porous base layer 11 can have an improved hydrophilicity, and both outer and inner portions thereof have a high affinity to water.

In the separating membrane 1 of the present disclosure, the hydrophilic substance 12 is selected from the group consisting of hyaluronic acid and its derivatives and water-soluble vitamins (e.g., vitamin C and B complex), and is preferably the hyaluronic acid. In this way, the separating membrane 1 of the present disclosure can absorb water to achieve a moldable softened state in a shorter period of time. However, without seriously impairing the expected effects of the present disclosure, other hydrophilic substances (such as substances containing a hydroxyl group, a carboxylic acid group, a sulfonic acid group, an ether group, an epoxy group, or an amine group) can also be used for the hydrophilization treatment in other embodiments.

A molecular weight of the hyaluronic acid that serves as the hydrophilic substance 12 of the present disclosure is not particularly limited, and is preferably between 10,000 daltons and 1,000,000 daltons and more preferably between 10,000 daltons and 700,000 daltons. If the molecular weight of the hyaluronic acid is less than 10,000 daltons, it is disadvantageous for the hyaluronic acid to be bonded to the biodegradable porous base layer 11. If the molecular weight of the hyaluronic acid is greater than 1,000,000 daltons, it is disadvantageous for the hyaluronic acid to penetrate into the biodegradable porous base layer 11.

More specifically, the hydrophilization treatment includes treating the biodegradable porous base layer 11 with an aqueous solution containing the hydrophilic substance 12, such that the hydrophilic substance 12 is bonded to the biodegradable porous base layer 11. The aqueous solution includes the hydrophilic substance 12 (e.g., hyaluronic acid) and water. Based on a total weight of the aqueous solution containing the hydrophilic substance 12 being 100 wt %, a content of the hydrophilic substance 12 can be from 25 wt % to 40 wt %. In practice, in a process of the hydrophilization treatment, the biodegradable porous base layer 11 is immersed in the aqueous solution containing the hydrophilic substance 12 for 30 seconds to 1 minute. However, the above description is for exemplary purposes only and is not intended to limit the scope of the present disclosure.

As shown in FIG. 2, the separating membrane 1 of the present disclosure can further include an outer covering layer 13. The outer covering layer 13 can cover either a portion of or all of the biodegradable porous base layer 11, and the hydrophilic substance 12 is present in the outer covering layer 13.

The separating membrane 1 of the present disclosure can be used in a bone repair surgery (e.g., a bone regeneration surgery) for rebuilding an underlying base structure before a dental implant. The separating membrane 1 of the present disclosure can separate an area for bone repair (i.e., an area filled with bone powders) from soft tissues, thereby preventing the soft tissues from growing into the area and affecting bone regeneration. It is worth mentioning that the separating membrane 1 of the present disclosure can be degraded and absorbed by the human body, and a secondary surgery is not required for removal thereof. Furthermore, compared to conventional separating membranes, the separating membrane 1 of the present disclosure has a better water wettability, a better coverability, and a better adhesive strength, and is more advantageous for surgical operations and repair, regeneration, and integration of a bone damaged area.

Specifically, a tensile stress of the separating membrane 1 of the present disclosure is from 0.3 MPa to 5 MPa at 25° C. and an absolute humidity of 50%. Furthermore, an adhesive strength (i.e., initial adhesion) of the separating membrane 1 of the present disclosure is from 0.3 N to 0.7 N according to the ASTM D3121-2006 standard.

It should be noted that, although the bone repair surgery is taken as an example in the present disclosure for describing features of the separating membrane 1, the separating membrane 1 can be used in other surgeries of the human body.

In the separating membrane 1 of the present disclosure, the biodegradable porous base layer 11 is mainly used to provide properties required for operations (e.g., mechanical strength and human absorption and metabolism). A material of the biodegradable porous base layer 11 includes a biodegradable polymer that can have a molecular weight ranging from 100,000 g/mol to 600,000 g/mol (preferably ranging from 150,000 g/mol to 350,000 g/mol). When biocompatibility is taken into consideration, the biodegradable polymer is preferably polylactic acid (PLA). In certain embodiments, based on a total weight of the material of the biodegradable porous base layer 11 being 100 wt %, a content of the polylactic acid can be greater than or equal to 50 wt %, and is preferably 100 wt %.

In addition, when the mechanical strength is taken into consideration, a thickness of the biodegradable porous base layer 11 can be from 200 lam to 400 μm. It is worth mentioning that within such a thickness range, the mechanical strength of the biodegradable porous base layer 11 is comparable to that of a PLA/PCL two-layered separating membrane. In certain embodiments, the thickness of the biodegradable porous base layer 11 can be 200 lam, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm.

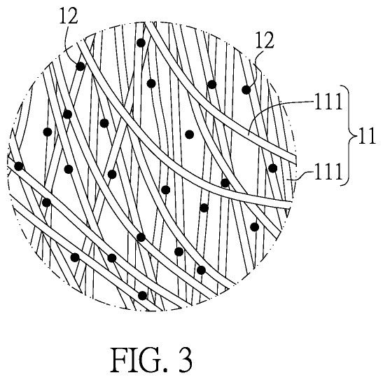

Referring to FIG. 3, an internal structure of the biodegradable porous base layer 11 is shown. As shown in FIG. 3, the biodegradable porous base layer 11 can be formed by electrospinning, and includes polylactic acid fibers 111. After undergoing the above-mentioned hydrophilization treatment, the polylactic acid fibers 111 are all adhered with the hydrophilic substance 12. It should be noted that, as long as water and nutrients are allowed to pass through, the internal structure of the biodegradable porous base layer 11 is not particularly limited.

### Second Embodiment

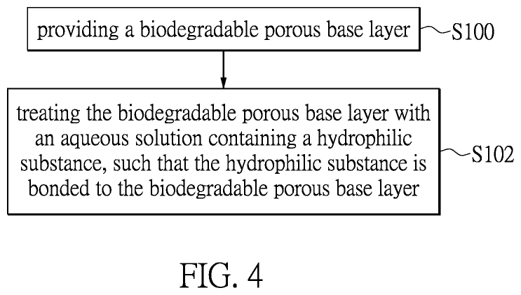

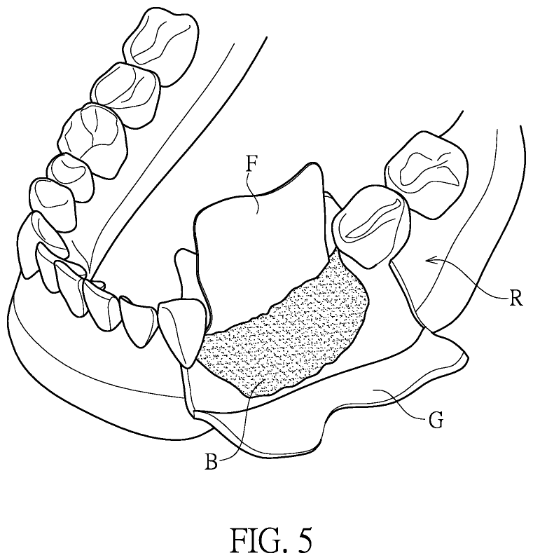

Referring to FIG. 4, a second embodiment of the present disclosure provides a method for manufacturing a separating membrane for dental surgeries, and said method can be used for manufacturing the separating membrane of the first embodiment. The method at least includes: providing a biodegradable porous base layer (step S100); and treating the biodegradable porous base layer with an aqueous solution containing a hydrophilic substance, such that the hydrophilic substance is bonded to the biodegradable porous base layer (step S102). In the following description, specific implementation details of each step will be described with reference to FIG. 1 to FIG. 3.

In step S100, the polylactic acid fibers 111 are provided and formed into the biodegradable porous base layer 11 in an electrospinning process.

A spinning solution for electrospinning can include polylactic acid and a solvent. An amount of the polylactic acid in the spinning solution can be from 1 wt % to 50 wt %, and an amount of the solvent in the spinning solution can be from 50 wt % to 99 wt %. In practice, the solvent can be selected from the group consisting of acetone, butanone, ethylene glycol, isopropanol, deacetylated chitin (DAC), N,N-dimethylformamide (DMF), dimethylacetamide (DMAC), dimethyl sulfoxide (DMSO), and ether.

In the electrospinning process, the spinning solution can be sprayed from a nozzle and solidified to form the polylactic acid fibers 111 in an electrostatic field, and then the polylactic acid fibers 111 are deposited on a collecting board. Through adjusting a movement of the nozzle, the polylactic acid fibers 111 can be tightly stacked, entangled, or interwoven along a specific direction, so as to form the biodegradable porous base layer 11 with a uniform thickness.

In step S102, the aqueous solution containing the hydrophilic substance 12 is used to treat the biodegradable porous base layer 11, such that the hydrophilic substance 12 is bonded to the biodegradable porous base layer 11. The aqueous solution includes the hydrophilic substance 12 and water. Specifically, the hydrophilic substance 12 not only adheres to the outer surface of the biodegradable porous base layer 11, but also penetrates into the biodegradable porous base layer 11. Accordingly, the polylactic acid fibers 111 are all adhered with the hydrophilic substance 12.

More specifically, based on the total weight of the aqueous solution containing the hydrophilic substance 12 being 100 wt %, the content of the hydrophilic substance 12 can be from 25 wt % to 40 wt %. The hydrophilic substance 12 is selected from the group consisting of hyaluronic acid and its derivatives and water-soluble vitamins, and is preferably the hyaluronic acid. In practice, in the process of the hydrophilization treatment, the biodegradable porous base layer 11 is immersed in the aqueous solution containing the hydrophilic substance 12 for 30 seconds to 1 minute. However, the above description is for exemplary purposes only and is not intended to limit the scope of the present disclosure.

As shown in FIG. 2, after step S102 is completed, the outer covering layer 13 covering the biodegradable porous base layer 11 is formed, and the hydrophilic substance 12 is present in the outer covering layer 13.

Relevant technical details mentioned in the first embodiment are still valid in the present embodiment and will not be repeated herein for the sake of brevity. Similarly, the technical details mentioned in the present embodiment can also be applied in the first embodiment.

A performance comparison between the separating membrane of the present disclosure and the conventional separating membranes currently on the market is as shown in Table 1 below.

TABLE 1

| | Separating | | | | | | | | | | | |
|---|---|---|---|---|---|---|---|---|---|---|---|---|
| | membrane of the | | | | | | | | | | | |
| | present disclosure | | Separating membranes currently on the market | | | | | | | | | |
| | Brand (model) | | | | | | | | | | | |
| | Nanya | | | | | | | | | | | |
| | bioresorbable | | Zimmer Biomet | | Nano Sigma | | Geistlich | | Ossix | | Curasan | |
| | membrane | | (OsseoGuard ®) | | Biotech | | (Bio-Gide ®) | | (Ossix Plus ®) | | (EpiGuide ®) | |
| Material | PLA membrane | | Crosslinked | | Uncrosslinked collagen | | | | | | PLA | |
| | adhered with HA | | collagen | | | | | | | | | |
| Thickness | 200 | um | 240 | μm | 100 | μm | 400 | μm | 260 | μm | 388 | μm |
| Tensile stress (dry) | 5.5 | Mpa | 29.1 | MPa | 2.18 | MPa | 4.4 | MPa | 5.3 | MPa | 0.6 | MPa |
| Tensile stress | 2.5 | Mpa | 5.7 | MPa | 1.5 | MPa | 3.4 | MPa | 2.9 | MPa | 0.5 | MPa |
| (wet) | | | | | | | | | | | | |
| Initial adhesion | No. 16/0.67N | | No. 8/0.33N | | No. 15/0.824N | | No. 12/0.48N | | — | | No. 7/0.1N | |
| test | | | | | | | | | | | | |
| (ball number/ | | | | | | | | | | | | |
| adhesive strength) | | | | | | | | | | | | |

In Table 1, different ball numbers indicate different steel ball sizes; the larger the ball number is, the higher the viscosity is.

In Table 1, the tensile stress (dry) refers to a tensile stress of the separating membrane/the conventional separating membrane that is tested at a temperature of 25° C. and an absolute humidity of 50%. The tensile stress (wet) refers to a tensile stress of the separating membrane/the conventional separating membrane that is tested after being immersed into a saline solution at 37° C. for minutes. The initial adhesion (adhesive strength) is tested according to the ASTM D3121-2006 standard.

### Beneficial Effects of the Embodiments

Therefore, in the separating membrane for dental surgeries and the method for manufacturing the same provided by the present disclosure, by virtue of “the hydrophilic substance being bonded to the biodegradable porous base layer, and being selected from the group consisting of hyaluronic acid, derivatives of the hyaluronic acid, and water-soluble vitamins” and “an outer surface of the separating membrane having a water contact angle of less than 80°,” an immersion and wetting time of the separating membrane can be greatly reduced before use (e.g., before surgical operations), and the fully-wetted separating membrane can have a better moldability, a better coverability, and a better adhesive strength.

More specifically, the separating membrane of the present disclosure can absorb water to achieve a moldable softened state within 5 minutes, so as to be adaptable for different three-dimensional shapes. Furthermore, the separating membrane of the present disclosure can securely adhere to a damaged area (e.g., a bone damaged area) and provide a sufficient growth space for the damaged area, thereby facilitating the repair, regeneration, and integration of the damaged area.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A separating membrane for dental surgeries, comprising a biodegradable porous base layer and a hydrophilic substance that is bonded to the biodegradable porous base layer, wherein the hydrophilic substance is selected from the group consisting of hyaluronic acid, derivatives of the hyaluronic acid, and water-soluble vitamins; wherein an outer surface of the separating membrane has a water contact angle of less than 80°.

2. The separating membrane according to claim 1, wherein a material of the biodegradable porous base layer includes polylactic acid, and the hydrophilic substance is hyaluronic acid having a molecular weight between 10,000 daltons and 700,000 daltons.

3. The separating membrane according to claim 2, wherein a thickness of the biodegradable porous base layer is from 200 μm to 400 μm.

4. The separating membrane according to claim 2, wherein the biodegradable porous base layer includes a plurality of polylactic acid fibers adhered with the hydrophilic substance.

5. The separating membrane according to claim 1, further comprising an outer covering layer, wherein the outer covering layer covers the biodegradable porous base layer, and the hydrophilic substance is present in the outer covering layer.

6. The separating membrane according to claim 1, wherein a tensile stress of the separating membrane is from 0.3 MPa to 5 MPa at 25° C. and an absolute humidity of 50%.

7. The separating membrane according to claim 1, wherein an adhesive strength of the separating membrane is from 0.3 N to 0.7 N according to the ASTM D3121-2006 standard.

8. A method for manufacturing a separating membrane for dental surgeries, comprising:
providing a biodegradable porous base layer; and
treating the biodegradable porous base layer with an aqueous solution containing a hydrophilic substance, such that the hydrophilic substance is bonded to the biodegradable porous base layer; wherein the hydrophilic substance is selected from the group consisting of hyaluronic acid, derivatives of the hyaluronic acid, and water-soluble vitamins;
wherein an outer surface of the separating membrane has a water contact angle of less than 80°.

9. The method according to claim 8, wherein a material of the biodegradable porous base layer includes polylactic acid, and the hydrophilic substance is hyaluronic acid having a molecular weight between 10,000 daltons and 700,000 daltons.

10. The method according to claim 9, wherein a thickness of the biodegradable porous base layer is from 200 μm to 400 μm.

11. The method according to claim 9, wherein, based on a total weight of the aqueous solution containing the hydrophilic substance being 100 wt %, a content of the hydrophilic substance is from 25 wt % to 40 wt %.

12. The method according to claim 11, wherein in the step of treating the biodegradable porous base layer with the aqueous solution containing the hydrophilic substance, the biodegradable porous base layer is immersed in the aqueous solution for 30 seconds to 1 minute.

13. The method according to claim 8, wherein, after the step of treating the biodegradable porous base layer with the aqueous solution containing the hydrophilic substance is completed, an outer covering layer is formed to cover the biodegradable porous base layer, and the hydrophilic substance is present in the outer covering layer.

14. The method according to claim 8, wherein in the step of providing the biodegradable porous base layer, a plurality of polylactic acid fibers are provided and formed into a layered structure by electrospinning; wherein, after the step of treating the biodegradable porous base layer with the aqueous solution containing the hydrophilic substance is completed, the plurality of polylactic acid fibers are adhered with the hydrophilic substance.
